(19) United States
(12) Patent Application Publication
(10) Pub. No.: US 2013/0323843 A1
(43) Pub. Date: Dec. 5, 2013

(54) POLYPHENOL PRODUCTION BY VACCINIUM MYRTILLUS CELL CULTURES

(71) Applicants: Sung-Yong H. Yoon, Lake Oswego, OR (US); Sonia Lall, Portland, OR (US)

(72) Inventors: Sung-Yong H. Yoon, Lake Oswego, OR (US); Sonia Lall, Portland, OR (US)

(73) Assignee: DIANAPLANTSCIENCES, S.A.S., Rennes (FR)

(21) Appl. No.: 13/983,537

(22) PCT Filed: Feb. 6, 2012
(86) PCT No.: PCT/US12/24008

§ 371 Date: Aug. 21, 2013

Related U.S. Application Data
(60) Provisional application No. 61/439,675, filed on Feb. 4, 2011.

(51) Int. Cl. C12N 5/04

(52) U.S. Cl. CPC C12N 5/04
USPC 435/420

(57) ABSTRACT

Cell cultures of Vaccinium myrtillus configured to grow in suspension culture in a liquid medium. The cells are derived from one or more V. myrtillus plant parts, such as an edible plant part (e.g., a leaf part or a berry part) or a stem part. The cells are adapted to grow to a high density is a relatively short period of time (e.g., about 7 days). In addition, the cells are adapted to produce high concentrations of polyphenols and/or procyanidins and essentially no anthocyanin. Methods for production of polyphenols and/or procyanidins from Vaccinium myrtillus cells grown in suspension culture are disclosed.

### BACKGROUND

The extract from the fruit Vaccinium myrtillus (more generally referred to as bilberry) has long been used for therapeutic purposes. In Europe it has been used for hundreds of years to treat diarrhea and dysentery, as well as diseases of the lungs, liver, and stomach. In addition, it is believed that British fighter pilots in World War II ate bilberry jam to help improve their night vision. More recently, extracts from the fruit of the V. myrtillus plant has been shown to possess potential anti-carcinogenic activity. The clinical benefits of V. myrtillus as both a dietary supplement and a therapeutic have been attributed to the presence of abundant amounts of flavonoids and anthocyanins in Bilberry. These antioxidant compounds scavenge damaging particles known as free radicals in the body, helping to prevent or reverse damage to cells. Antioxidants have been shown to help prevent a number of long-term illnesses such as heart disease, cancer, and macular degeneration. The V. myrtillus fruit also contains tannins, which are known to act as both an anti-inflammatory and an astringent.

Polyphenols are widely distributed in plants, fruits, and vegetables and have received considerable attention because of their physiological functions in human and animal health, including antioxidant, antimutagenic and cancer prevention activities (Salvia et al., J. Agric. Food Chem. 39: 1549-1552, 1991; Bomser et al., Cancer Lett., 135: 151-157, 1999; Zhao et al., Carcinogenesis, 20: 1737-1745, 1999). Epidemiological studies have suggested that flavonoids, among the polyphenols, may reduce the risk of heart disease (Hertog et al., Lancet: 342: 1007-1011, 1993). Additionally, dietary flavan-3-ols and/or proanthocyanidins have been shown to reduce the incidence of atherosclerosis and coronary heart disease in experimental animals (Tijburg et al., Atherosclorosis, 135: 37-47, 1997; Yamakoshi et al., Atherosclerosis, 142: 139-149, 1999). One of the mechanisms responsible for these effects involves their inhibition of oxidation of low density lipoprotein (LDL) (Steinberg, Circulation, 85: 2337-2344, 1992).

Berries of the Vaccinium species have been shown to possess radical scavenging capacity in various in vitro models using assays of the oxygen radical absorbance capacities (ORAC), the ferric reducing antioxidant power (FRAP), the total oxidant scavenging capacity (TOSC), and the free radical scavenging activity against 2,2-diphenyl-1 -picrylhydrazyl (DPPH) radical as well as antioxidant capacities in inhibiting oxidation of methyl linoleate, liposomes, and human low-density lipoprotein (LDL) (Maata-Riihinen et al). Cultivated cranberry (V. macrocarpon Ait.) and wild lingonberry contain both A- and B-type procyanidins (Gu et.al., Morimoto et.al., Foo et.al.) whereas primary B-type procyanidins were identified in wild (V. angustifolium Ait.) and cultivated blueberries (V. corymbosum L., V. ashei L.) (Foo et.al.; Prior et. al.; Schmidt et.al.) Rare A-type low molecular weight procyanidins were detected in wild lingonberry, cranberry, bilberry, and bog whortleberry and were present at higher levels than the more common B-type procyanidins (Maata-Riihinen et.al.). The rare A-Type procyanidin is known to act as deterrent to adhesion of bacterial cilia to the endothelial layers helping in prevention of urinary tract infection (Nowack and Schmidt; Foo et al²;). It is a general anti inflammatory agent which is known in Bilberry for many decades to cure intestinal inflammations. In addition, V. myrtillus leaves have 35 different flavon-3-ols, procyanidins, flavonols and their glycosides, and various phenolic acid conjugates (Hokannen et.al.).

Vaccinium myrtillus is difficult to grow and is therefore rarely cultivated. As a result, the fruit is generally collected from wild plants during its limited growing season (May through September), which must be both wet and warm. Thus, the supply of the berries is unreliable and the berries are available in limited quantities. Moreover, the fruit are softer and juicier than the related blueberry, such that they must be harvested by hand, and are difficult to transport, which contribute to the high cost of the fresh fruit harvested from the V. myrtillus plant. Also due to the high demand of the ripe fruit, unripe fruits and leaves are not economically viable products to collect. These are the parts of the plant that have highest amounts of the procyanidin. In view of the clinical benefits of V. myrtillus and the difficulty in cultivating these plants, there is a need to develop a sustainable in vitro culture system for the cells of these plants.

### SUMMARY

The present disclosure relates to cell culture of Vaccinium myrtillus that are configured to grow in suspension culture in a liquid medium. The cells are derived from one or more V. myrtillus plant parts, such as an edible plant part (e.g., a leaf part or a berry part) or a stem part. The cells are adapted to grow to a high density in a relatively short period of time (e.g., about 7 days). In addition, the cells are adapted to produce high concentrations of polyphenols and/or procyanidins and essentially no anthocyanin. Methods for production of polyphenols and procyanidins from Vaccinium myrtillus cells grown in suspension culture are also disclosed.

In one embodiment, a cell culture is described. The cell culture includes a plurality of friable Vaccinium myrtillus cells in a suspension cell culture. The cells in suspension culture are derived from one or more of: a hypocotyl, a cotyledon, a leaf section, a stem section, or a root section of a seedling; or a berry, a stem section including a node or an internode, or a leaf section of a mature plant. In one embodiment, the cells can be derived from an edible plant part, such as a leaf part or a berry part. The cells are selected to be capable of obtaining a packed cell volume of at least 55% in 7 days of growth, wherein at least 10% of a dry mass of the plurality of Vaccinium myrtillus cells is comprised of polyphenols and at least 5% of a dry mass of the plurality of Vaccinium myrtillus cells is comprised of procyanidins.

Preferably, at least 12.5%, 15%, 20%, or more of the dry mass of the plurality of Vaccinium myrtillus cells is comprised of polyphenols. Preferably, at least 7.5%, 10%, 15%, 20%, or more of the dry mass of the plurality of Vaccinium myrtillus cells is comprised of procyanidins. It is also preferred that the mass of cells is essentially free of anthocyanins. For example, it is preferred that the dry mass of the plurality of Vaccinium myrtillus cells includes less than 0.5%, 0.1%, 0.01%, 0.001%, or less anthocyanin.

In another embodiment, a method of producing a cell culture of Vaccinium myrtillus cells is described. The method includes (1) producing a cell callus of Vaccinium myrtillus cells derived from one or more of: a hypocotyl, a cotyledon, a leaf section, a stem section, or a root section of a seedling; or a berry, a stem section including a node or an internode, or a leaf section of a mature plant, (2) introducing one or more cells derived from the callus into a liquid medium, (3) agitating the one or more cells in the liquid medium, (4) replacing the liquid medium with a fresh liquid medium or transferring the cells to fresh a fresh liquid medium to establish a suspension cell culture of Vaccinium myrtillus, (5) growing the suspension cell culture of Vaccinium myrtillus to a packed cell volume of at least 55%, and (6) selecting suspension cell cultures having at least 10% of a dry mass of the plurality of Vaccinium myrtillus cells comprised of polyphenols and/or at least 5% of a dry mass of the cells comprised of procyanidins.

In yet another embodiment, a method of increasing growth of Vaccinium myrtillus cells in suspension cell culture is described. The method includes (1) providing a suspension cell culture of Vaccinium myrtillus cells, (2) culturing the cells in a liquid medium in suspension culture, and (3) selecting suspension cell cultures having greater than 45% packed cell volume (PCV).

In one embodiment, the method of increasing growth of Vaccinium myrtillus cells in suspension cell culture further includes selecting suspension cell cultures having increased polyphenol and procyanidin accumulation in response to increased sugar concentration in the liquid medium. In one embodiment, the sugar concentration in the liquid medium includes approximately 30-60 g/L sucrose. In one embodiment, procyanidin accumulation in the cells in suspension culture is increased from about 1-2 g/L of PCV at 20 g/L sucrose to about 3-7 g/L of PCV at 30 g/L sucrose. In one embodiment, polyphenol accumulation in the cells in suspension culture is increased from about 2-4 g/L of PCV at 20g/L sucrose to about 5-10 g/L of PCV at 60 g/L sucrose.

In still yet another embodiment, a method of increasing polyphenol production from Vaccinium myrtillus cells in culture is described. The method includes (1) selecting a plurality of Vaccinium myrtillus cells adapted to grow in suspension culture, (2) and culturing the cells in suspension culture in the presence of a sufficient amount of sugar to increase polyphenol production.

In one embodiment, the sufficient amount of sugar is the liquid medium having greater than 20 g/L sugar, 20 g/L to 30 g/L sugar, or greater than 30 g/L sugar. In one embodiment, the sugar is sucrose. In another embodiment, the sugar is glucose. In one embodiment, the sugar is present in an amount sufficient for polyphenol production to increase above 3 g/L packed cell volume (PCV). In another embodiment, the sugar is present in an amount sufficient for polyphenol production to increase to at least 7 g/L packed cell volume (PCV).

In still yet another embodiment, a method of extracting polyphenols from Vaccinium myrtillus cells in culture is described. The method includes (1) selecting a plurality of Vaccinium myrtillus cells adapted to grow in suspension culture, and (2) extracting polyphenols from the cells using a solvent, wherein at least 10% of a dry mass of the plurality of Vaccinium myrtillus cells is comprised of polyphenols and at least 5% of a dry mass of the plurality of Vaccinium myrtillus cells is comprised of procyanidins.

In one embodiment, the solvent includes acetone, acetic acid, and water. In one embodiment, the solvent includes 70% acetone (v/v) and 0.5% acetic acid (v/v).

These and other objects and features of the present disclosure will become more fully apparent from the following description and appended claims, or may be learned by the practice of the claims as set forth hereinafter.

### BRIEF DESCRIPTION OF THE DRAWINGS

To further clarify the above and other advantages and features of the present disclosure, a more particular description of the subject matter of the disclosure will be rendered by reference to specific embodiments thereof which are illustrated in the appended drawings. It is appreciated that these drawings depict only illustrated embodiments of the disclosure and are therefore not to be considered limiting of its scope. The subject matter of the disclosure will be described and explained with additional specificity and detail through the use of the accompanying drawings in which:

FIG. 1 shows the consumption of sugar with increasing biomass from 25% initial biomass to 50% in one week.

FIG. 2 shows the growth (a) RI (b) and production (c) at different shaker speeds. The 500 ml flasks were all inoculated at 20% PCV and PCV, RI and production yield was measured after 6 days of growth.

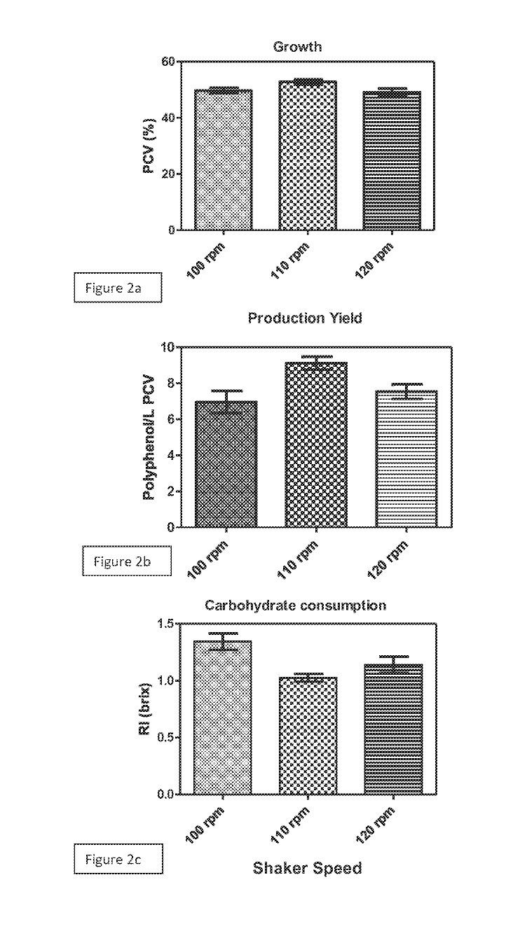

FIG. 3 shows the HPLC chromatogram of extracts from suspension cells derived from stem, hypocotyl, leaf and cotyledon explants in fluorescence detector mode. The labels 1 through 12 indicate the degree of polymerization of procyanidins, respectively: 1, monomers; 2, dimers; 3, trimers; 4, tetramers; 5, pentamers; 6, hexamers; 7, heptamers; 8, octamers; 9, nonamers.

FIG. 4 shows the HPLC chromatogram of extracts from suspension cells of Bilberry and cocoa in fluorescence detector mode. The labels 1 through 12 indicate the degree of polymerization of procyanidins, respectively: 1, monomers; 2, dimers; 3, trimers; 4, tetramers; 5, pentamers; 6, hexamers; 7, heptamers; 8, octamers; 9, nonamers. This figure confirms that the peaks in Bilberry are procyanidin oligomers by the fact that the 2 cell lines were extracted in the same way and are run under same conditions and they have same retention time for each oligomer.

FIG. 5 shows a UV absorption pattern at 280 nm of cocoa (a) and bilberry (b) extracts confirming the presence and detection of procyanidins in Bilberry.

### DETAILED DESCRIPTION

The present disclosure relates to cell culture of Vaccinium myrtillus that are configured to grow in suspension culture in a liquid medium. The cells are derived from one or more V. myrtillus plant parts, such as an edible plant part (e.g., a leaf part or a berry part) or a stem part. The cells are adapted to grow to a high density is a relatively short period of time (e.g., about 7 days). In addition, the cells are adapted to produce high concentrations of polyphenols and/or procyanidins and essentially no anthocyanin. The subject matter of the disclosure will be described and explained with additional specificity and detail through the use of the following Examples.

### EXAMPLES

### Example 1

### Surface Sterilization and Seed Germination

Vaccinium myrtillus seeds were obtained from Horizon Herbs, Oregon. Leaves, stem sections and immature berries of V. myrtillus (Erin's Bilberry) used in this Example and the Examples below were collected from National Clonal Germplasm Repository (NCGR) in Corvallis, Ore.

Leaves, stem sections, and immature berries were rinsed in running water for 20 minutes and rinsed in 75% ethanol for 1 minute. Stems were then cut into smaller pieces. Then the stems, leaves and immature berries were washed in 25% sodium hypochlorite (v/v) for 15 minutes followed by 5 rinses in sterile distilled water.

Seeds (Horizon Herbs, Oregon) were surface sterilized by rinsing first, in 75% ethanol for 1 minute. Then they were washed in 25% sodium hypochlorite (v/v) for 15 minutes followed by 5 rinses in sterile distilled water. Seeds were then suspended in 0.1% agarose and plated onto 100×25 mm Petri plates (approximately 100 seeds per plate). They were germinated on MS (Murashige and Skoog) medium (4.43 g/L) with 7g/L agar under a 16 hour light and 8 hour dark photoperiod at 23° C. for 4 weeks.

### Example 2

### Callus Induction from Vaccinium myrtillus Seedlings Grown In Vitro

More proliferative growth and friable callus are very important characteristics of a successful cell line. This example describes methods and media conditions which were optimized to initiate and maintain callus from various explants derived from in vitro grown V. myrtillus seedlings.

Callus was initiated from hypocotyls, cotyledons, leaves, stem sections and roots of in vitro grown seedlings. Plant parts were cut into 5mm sections. All media were sterilized by autoclaving for 20 minutes at 121° C. and 15 PSI (pounds per square inch) unless otherwise stated. All growth regulators were filter sterilized and added post autoclave unless otherwise stated. All cultures were kept in darkness at 25° C. unless otherwise stated.

Explants were put on various callus induction media (Table 1). Plates were kept in darkness at 25° C. First signs of callus formation were seen after 2 weeks of putting explants on plates with media VM1445, VM1196, VM1204, and VM1233 (VM1233 is described in Madhavi et al., Plant Science, 131:95-103, 1998). Callus induction rates were 83%, 85%, 85% and 70% respectively. However, callus produced on media VM1196 and VM1204, both of which had 24 mM ammonium sulfate and 8 mM potassium nitrate but different base salts, (MS basal salts no nitrogen and B5 major salts modified, respectively; Table 1)was softer than callus produced on medium with 1mM ammonium sulfate and 24 mM potassium nitrate (VM1445). Callus produced on medium VM1233 (Madhavi et al., Plant Science, 131:95-103, 1998) was very compact and non proliferative. Madhavi et al. showed that callus was subcultured on this medium for three subculture periods each at three week intervals, although the quality of the callus on this medium was not discussed in that reference. Subculturing callus using the conditions of Madhavi et al. produced the same initial results as those described in the reference, but it was noted that with every subculture the callus became hard and non proliferative. Thus, the quality of the callus using medium VM1233 decreased with every subculture. The removal of the polyvinylpyrrolidone (PVP; medium VM1204) from the VM1233 medium helped to make the callus soft. On media VM1491 and DC1152, 50% and 10% of the explants produced sustainable callus, respectively. Explants and calli were transferred to fresh medium every 3 weeks. Once the calli were separated from explants, calli that were very proliferative were subcultured every 2 weeks. Fast growing cell lines were chosen for subculture. Continuous subculture helped change the morphology of the callus to a more desirable friable morphology.

Subculturing continuously on medium with higher ammonium sulfate and lower potassium nitrate resulted in the callus becoming very brown from the stress and it eventually stopped growing. This was evident from the fact that medium VM1445, which had full strength Gamborg's B5 (B5) medium, but did not have ammonium sulfate or potassium nitrate did not show browning and eventual death. Media VM1196 and VM1204 were discontinued after 9 weeks because of undesired browning of the callus. Various media (Table 1) were tried in order to characterize a medium that would support growth of callus sustainably, without browning and eventual death. Medium VM1516 which had full strength MS salts showed very proliferative and sustainable growth. When VM1445 and VM1516 were compared, VM1516 gave the most proliferative calli and also helped change the morphology from compact to granular and eventually friable callus. Medium VM1516 also proved the best for sustainably maintaining callus derived from V. myrtillus seedlings. VM1516 was also confirmed to be the best medium for initiating new callus from various V. myrtillus seedling explants, with a success rate of 83%.

### Example 3

### Callus Induction from Vaccinium myrtillus Tissue Collected from NCGR

This example describes methods and media formulations for initiating and maintaining callus from various explants (derived from berries, nodes, internodes, or leaves) derived from field-grown V. myrtillus plants.

Mature leaves and stems, and immature berries were surface sterilized, as discussed above. The plant parts were cut into small 5 mm sections before explanting into media VM1516 and VM1491. Berries were cut open under sterile conditions and the skin was placed on culture plates with media. Any berry flesh was removed before explanting.

Culture plates were kept in darkness at 25° C. In general, callus was observed 4 weeks after initially explanting materials on VM1516 and 6 weeks on VM1491. With regard to leaf explants, there was a 53% callus induction rate overall. Callus from leaf explants was produced in VM1491 (73% of initial explants) and in medium VM1516 (76% of initial explants) and no callus was observed in medium VM1672 and TC1596. It was observed that the callus produced in VM1516 was more vigorous than that produced in VM1491. With regard to nodes, 47% of those explants produced callus in VM1516. Internodes were placed on 3 different media VM1516, VM1491 and TC1596. On VM1516, 51% explants produced callus, while only 20% produced callus on VM1491 and none on TC1596. Among the explants from berries, 59% produced callus on VM1516.

Callus derived from V. myrtillus tissue was subcultured every 3 weeks on VM1516. This callus was very proliferative and friable cell lines were selected for further maintenance. Calli derived from these tissues were maintained on medium VM1516 for over eight months and have demonstrated consistent proliferation without change in quality of the callus.

### Example 4

### Suspension Creation from Callus Derived from Vaccinium myrtillus Seedlings

Friable cell lines created as in example 2 were chosen for initiation of suspensions. Cell suspensions were created by introducing 1 g (approx) of fresh 2 week old V. myrtillus seedling callus (prepared as in Example 2) into 15 ml of liquid medium (VM1799, VM1831 or DC1151; Table 2) in a sterile 125 ml Erlenmeyer flask. The flasks were covered with sterile silicon (foam) caps and agitated at 120 revolutions per minute (rpm) in a gyrotatory shaker. The suspensions were kept in darkness at 23° C. To establish the cell culture, the spent medium was removed and fresh medium was added every week for 2 subcultures. The growth of cells was measured by the rate of carbohydrate consumed by measuring the delta of refractive index (RI) (as measured by degrees of BRIX (i.e., % BRIX)) of the medium. If the RI was less than or equal to half of the initial RI of the medium, fresh medium was added to the cells. If the RI was greater than half, fresh medium was only added after 2 weeks. The subcultures were transferred weekly or biweekly as deemed necessary.

Cultures that formed as either granular or fine suspension of cells were retained, while cultures that did not form suspension cultures were discarded. Once the suspension culture was established (3-4 subculture periods), 25-35% of the cells were transferred to flask with fresh medium every week. Packed cell volume (PCV) and RI was recorded at every subculture to measure cell growth.

Sustainable stable suspensions were obtained within 6 subcultures of initiating suspensions from callus.

### Example 5

### Suspension Creation from Callus Derived from Vaccinium myrtillus Tissue Collected from NCGR

Friable cell lines were chosen for initiation of suspensions. Cell suspensions were created by introducing V. myrtillus callus (prepared as in Example 3 from nodes, internodes, leaves, and berries) into liquid medium (VM1933; Table 2) in sterile Erlenmeyer flasks. The flasks were covered with sterile silicon (foam) caps and agitated at 120 revolutions per minute (rpm) in a gyrotatory shaker. The suspensions were kept in darkness at 25° C. To establish the cell culture, the spent medium was removed and fresh VM1933 medium was added. The growth of cells was measured by the rate of carbohydrate consumed by measuring the delta of refractive index (RI) of the medium. If the RI was less than or equal to half of the initial RI of the medium, fresh medium was added to the cells. If the RI was greater than half, fresh medium was only added after 2 weeks.

### Example 6

### Optimization of Cell Growth

This example describes methods used to increase cell growth of suspensions. Cell culture productivity increases as a function of the rate of cell growth and the density at which cell growth stops. To determine the optimal inoculation density, suspension cultures of Vaccinium myrtillus cells were initiated with an inoculum size yielding a starting cell density of 15% packed cell volume (“PCV”) and 25% PCV and allowed to grow for 7 days. Cultures initiated at a cell density of 15% PCV did not reach maximal density within 7 days. Cultures initiated at a cell density of 25% PCV in Medium VM1831 (Table 1.) doubled in density (i.e., total cell volume) within 7 days and reached a maximal average cell density of 45-50% PCV within 7 days with some cell line cultures showed over 55-60% PCV at day 7. Cell selection helped to capture cultures that reached a 45% PCV or more PCV within 7 days or less (a rapidly growing cell culture). Cultures that took more than 7 days to reach 45% PCV were discarded.

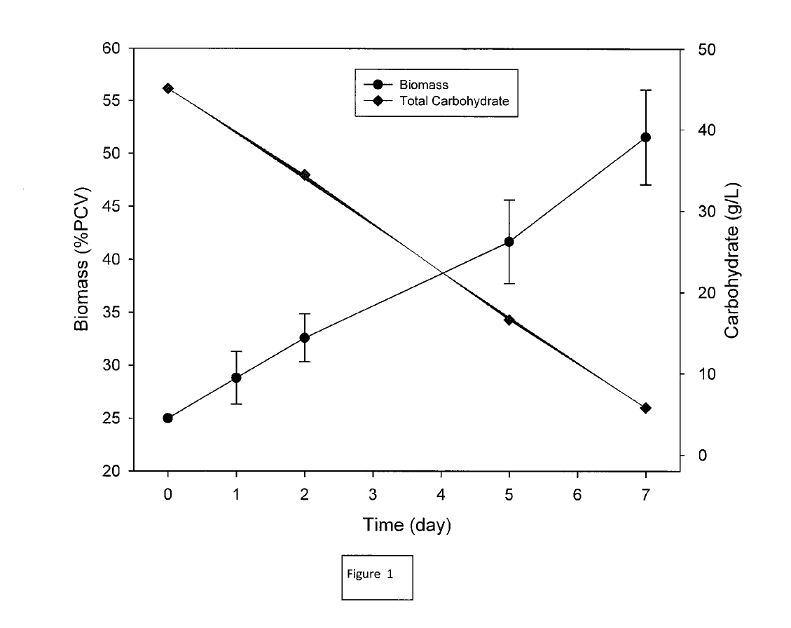

After careful selection of cells that showed high sugar consumption and good growth (FIG. 1.) through a number of generations, it was seen that the final cell density on day 7 was very high (PCV around 65.8±0.63) when flasks were initiated with a 25% inoculum. This inhibited proper shaking of the cells in the 125 ml flasks with a working volume of 40 ml. Therefore, optimization of inoculums size was required again once the cell line improved through cell selection process. Calculation of the doubling time indicated that a 20% PCV inoculums would yield around 60% final PCV on day 7. Experimental data supported this and showed there was a significant difference (P=0.008) in the final PCV (59.9±0.77) but no deleterious effect (P=0.39) on production or productivity. Hence the inoculums size was changed to 20% PCV.

### Example 7

### Optimization of Polyphenol Production from Bilberry Suspensions by Cell Selection and Medium Optimization of Suspension Cell Culture

After optimization of growth, production of polyphenol production was achieved by changing media formulation and additional criteria for cell selection.

The carbohydrate consumption was rapid in the cultures with the cultures reaching RI of 0 to 0.6 by day 7. Polyphenol and/or procyanidin production in VM1831 was low possibly due to sugar starvation. The medium VM1831 had 20 g/L of sucrose. Liquid media was optimized by adjusting carbohydrate level to maintain cultures without nutrient starvation. A new medium VM1933 (Table 1.) was formulated with 30 g/L of sucrose to avoid sugar starvation of the cells. In this medium the RI went down to between 0.8 and 1.0. The production values of polyphenols went up from about 2-4 g/L of PCV at 20g/L sucrose to about 5-10 g/L of PCV at 60 g/L sucrose within 4 subcultures and could be maintained at a high production level. The production values of procyanidins went up from about 1-2 g/L of PCV at 20 g/L sucrose to about 3-7 g/L of PCV at 30 g/L sucrose within 4 subcultures and could be maintained at a high production level.

The cell selection process where we selected for flasks that produced higher than average polyphenols quantified by a high through at each subculture allowed for further improvement in polyphenol and procyanidin production levels.

### Example 8

### Detection and Confirmation of Polyphenol and Procyanidin Production in Suspensions from Various Parts of Bilberry Seedlings

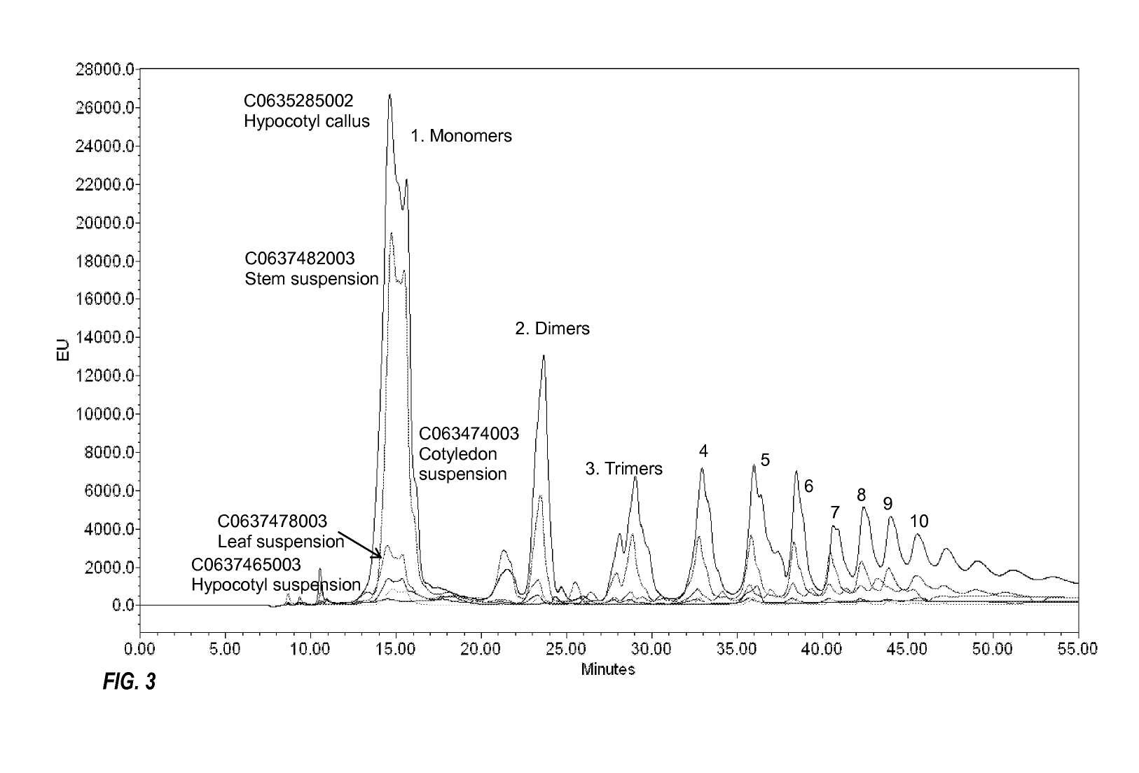

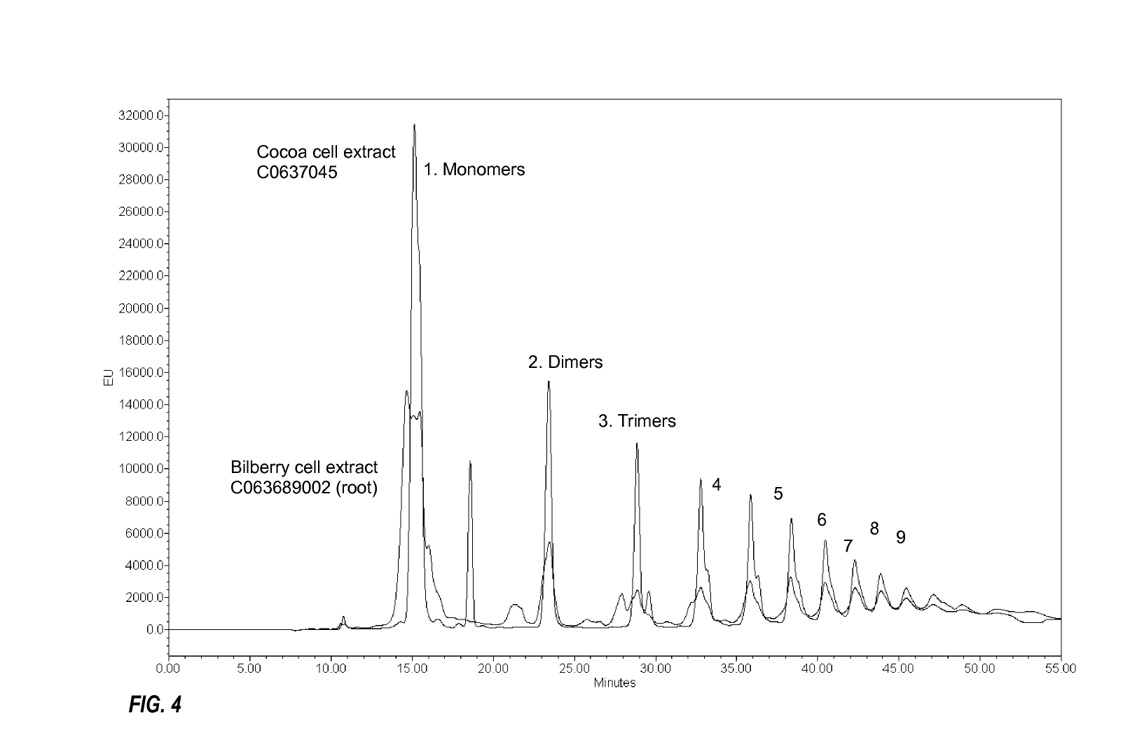

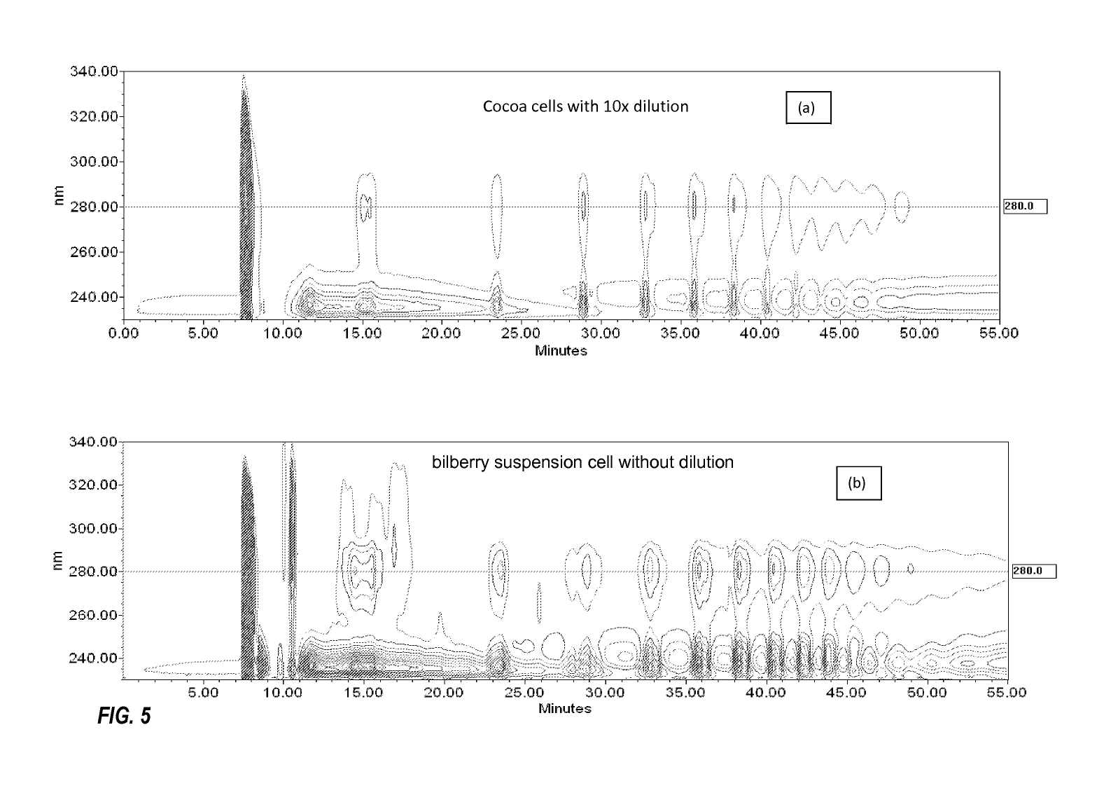

In this example we demonstrate that we have been able to produce procyanidin from suspensions prepared as in examples 4 and 5 from all parts (roots, hypocotyls, berries, cotyledons, stem and leaves) of the plants. FIG. 3 shows the chromatogram showing various sources. We have also been able to confirm that what we are seeing is procyanidins by overlaying with confirmed cocoa procyanidin chromatograms (FIG. 4) that show same retention times for each oligomer as in cocoa, which also show additional isomers of dimer, trimer and tetramer in Bilberry. Also running a UV absorption at 280 nm showed that the pattern was similar to cocoa and also confirmed presence of procyanidin (FIG. 5).

### Example 9

### Extraction of Polyphenols from Vaccinium Callus Culture and Suspension Cultures

This example describes methods developed for extracting polyphenols from callus and suspension cells of Vaccinium cultures developed in examples 1-5. Polyphenols were extracted from approximately 0.4 ml of fresh cells from suspensions with 0.4 ml 70% (v/v) acetone with 0.5% acetic acid. A robust high throughput method was used as follows: From each flask of cell culture to be analyzed, the packed cell volume (PCV) of the sample was recorded prior to transferring 0.4 ml into a 96- deep well plate. The supernatant from each well was removed and discarded with a plastic transfer pipette. Next, 0.4 ml of extraction solvent (70% acetone, 29.5% water, 0.5% acetic acid) and a tungsten carbide bead were added to each well, and the plate was placed on a Mixer Mill to grind the cells at 18 Hz for 4 minutes. The plate was then placed in a centrifuge and centrifuged at 6000 rpm for 4 minutes to separate the cells from the extract.

### Example 10

### Preliminary Analysis of Polyphenol Production in Culture

The method used to carry out the procyanidin analysis reaction was designed to approximate fairly closely the original Swain and Hillis (J. SCI. Food Agric. 10:63, 1959) method and Porter et al. (Phytochemistry, 25(1):223, 1986) method. The butanol-HCl extraction assay was used to measure polyphenols in the extracts of Vaccinium myrtillus suspended cells. The polyphenols are hydrolyzed to the monomers of (−)-epicatechin and cyanidin by combining 0.1 ml of aqueous acetone extract and 1.0 ml of butanol-HCl reagent (95:5 v/v) and heating the solution at 75° C. for 60 minutes in a Qiagen deep well block (Valencia, Calif., USA). Presence of cyanidin in the hydrolyzed sample was observed by the formation of a pink color. The absorbance at 520 nm was determined, and procyanidin content was calculated based on the amount of cyanidin formed using a calibration curve created using different concentrations of procyanidin B2 purchased from Chromadex, Inc. (Irvine, Calif.). Brighter pink color indicated higher concentration of procyanidins in suspension cultures. Based on this method the procyanidin content of several suspension cultures ranged from 1 g/L to 10 g/L.

Total polyphenol content of bilberry cell extracts was measured using the Folin-Ciocalteau assay (Slinkard, K.; Singleton, V. L. Total Phenol Analysis:Automation and Comparison with Manual Methods. American Journal of Enology and Viticulture 1977, 28: 49-55). Cell culture extracts, in 70% acetone with 0.5% acetic acid, were analyzed for total polyphenol content by taking 25 μl extract and adding it to 0.975 ml of water to dilute the sample prior to beginning the assay. For the quantification of polyphenols, 20 μl of the diluted extract is added to 0.790 ml water plus 50 μl of Folin-Ciocalteau reagent. The reaction is then stopped by the addition of 150 μl sodium carbonate solution. The resulting solution is measured at 765 nm and compared to a calibration curve of various dilutions of gallic acid solution measured by the same assay to determine the concentration of total polyphenols in the cell extracts.

### Example 11

### Small Scale Extraction of Polyphenols from Fresh Bilberry Cells or Ground Freeze Dried Cells

Bilberry cells (0.5 mL) without media or 50 mg of ground bilberry cells were sampled in 2.0 ml of micro-tubes or 1.2 ml tubes in a 96 well block from Qiagen, Inc. Appropriate volumes of acidic (0-2% of citric, acetic or ascorbic) aqueous extraction solvent (30-80% of acetone, ethanol, methanol) was added to each of the bilberry cell samples and then placed into ultrasonicator or BeadMill to extract polyphenols and/or procyanidins. The samples were centrifuged for 4 minutes at 6000 rpm (RCF 5996). The supernatant may be filtered with 0.45 um membrane filter and diluted to 10× (if necessary) by using the same aqueous extraction solvents prior to analysis. The leftover extracts were stored in −20 degree of freezer for further analyses.

### Example 12

### Analysis of Procyanidin Production in Bilberry Culture

LC analyses were performed on the Bilberry cell extracts using a Waters (Milford, Mass., USA) Alliance HPLC system equipped with a CTC Analytics PAL autosampler (Leap Technologies, Carrboro, N.C., USA), Waters 626 pump with 600S Controller and a Waters 2996 photodiode-array detector (PDA) scanning from 190 to 780 nm. Gradient elution was carried out with water-0.1% formic acid (solvent A) and acetonitrile-0.1% formic acid (solvent B) at a constant flow-rate of 0.3 ml/ minute. A linear gradient with the following proportions (v/v) of solvent B was applied (t(min), % B): (0, 7), (5, 15), (20, 75), (25, 100), (35, 100), (35.1, 7) (45, 7). The column was Ultra Aqueous C18 column (100×2.1 mm i.d., 3.5 μm) (Restek, Bellefonte, Pa. USA). The procyanidin monomers of (+)-catechin, (−)-epicatechin, and oligomeric procyanidins (dimer to hexamer) were monitored at 280 nm. A Waters Quattro Micro triplequadrupole mass detector (Milford, Mass., USA) was used to obtain the MS data and analyzed by MassLynx™ software. Full-scan data acquisition was performed, scanning from m/z 150 to 1800. Authentic standards for catechin, epicatechin, were purchased from Sigma-Aldrich, Inc. (St. Louis, Mo.) and dilutions made to create calibration curves in order to detect and quantify the metabolites.

Analysis of quantifiable procyanidins was performed by normal phase HPLC system consisted of the Waters 2795 separation module, the Waters 996 PDA detector and the Waters 474 scanning fluorescence detector. Characterization and separation conditions of procyanidins in bilberry cell extracts obtained using Develosil Diol (250×4.6 mm ID, 5μ, particle size) adapted from Kelm et al., the improved process for analyzing for separating, and for isolating polar protic monomers and/or oligomers. (U.S. Pat. No. 0,075,020). The binary mobile phase consists of solvent (A), acetonitrile: acetic acid (98:2, v/v) and solvent (B), methanol: water: acetic acid (95:3:2, v/v/v). A linear gradient elution was performed at 30° C. with 0.8 mL/min flow rate as follows: 0-35 min, 100-60% A; 35-40 min, 60% A; 40-45 min, 60 - 100%A. Separations of oligomer procyanidins were monitored by fluorescence detection (excitation wavelength at 276 nm, emission wavelength at 316 nm), UV detection at 280 nm (FIG. 10A). (Lazarus et al. J. Agric. Food Chem. 47 (1999), 3693) and PDA (FIG. 10B).

The purpose of the analytical method is to detect the presence of the ten different individual procyanidins in fresh bilberry cells or freeze-dried cells. Detectable procyanidins are monomer, dimmers, trimers, tetramers, pentamers, hexamers, heptamers, octamers, nonamers and decamers.

The samples prepared from fresh bilberry cells, freeze-dried bilberry cells and bilberry cell extract were analyzed for procyanidin estimation using internally prepared procyanidin standards from cocoa beans by executing internal HPLC method on Empower 2.

### Example 13

### Scale-up of Bilberry Suspension Culture

A common problem in the use of plant cell cultures is obtaining consistent production of target products (Kim et al., Biotechnol Prog. 20(6) 1666, 2004). Therefore, a key for successful large-scale plant cell culture is to maintain stable productivity. A process to scale-up suspensions of bilberry cell cultures from 125 ml flasks to 250 mls and then 500 ml flasks was successfully conducted. The speed of the shakers was optimized for 500 ml flasks to give the same kind of growth and production numbers as in the 125 ml flasks. Three different shaker speeds were tested—100, 110 and 120 RPM. The average PCV was 50˜55% at seven days, which was about 2.5 times greater than the initial PCV level of 20% for all the treatments. However, the production yield (PY) was significantly high at 110 RPM when compared to 100 RPM with a P value of 0.005. Although the difference in PY was not significant between 110 RPM and 120 RPM, the color in the 120 RPM flasks was slightly darker, leading to choose 110 RPM as preferred shaker speed for 500 ml flasks. Every seven days of culture, biomass, sugar concentration in medium, and polyphenol and/or procyanidin productivity, were measured.

Feasibility of scale up to 2.8 L flasks is carried out, where shaking speed (rpm) and shaker stroke size is optimized. This successfully yields similar growth and production as in 125 ml and 500 ml flasks.

TABLE 1

| | VM1196 | VM1204 | VM1445 | VM1222 | VM1233 | VM1491 | VM1672 |
|---|---|---|---|---|---|---|---|
| MS salts (g/L) | | | | | | | 4.33 |
| (Phytotech | | | | | | | |
| Catalog # M524) | | | | | | | |
| (g/L) | | | | | | | |
| Gamborg's B5 Salts | | | 3.1 | | | 3.1 | |
| (g/L) | | | | | | | |
| (Phytotech Catalog # | | | | | | | |
| G768) | | | | | | | |
| MS basal salts no | 0.788 | | | | | | |
| nitrogen (g/L) | | | | | | | |
| (Phytotech | | | | | | | |
| Catalog # M531) | | | | | | | |
| B5 major salts | | 50 | | 50 | 50 | | |
| modified¹ (ml/L) | | | | | | | |
| (20x) (EPS 000210 - | | | | | | | |
| Table 6) | | | | | | | |
| B5 minor salts (ml/L) | | 1 | | 1 | 1 | | |
| (1000x) (EPS 0004 - | | | | | | | |
| Table 7) | | | | | | | |
| MS vitamins (mL/L) | | | | | | | |
| 1000X Stock | | | | | | | |
| Solution | | | | | | | |
| (Phytotech | | | | | | | |
| Catalog # M533) | | | | | | | |
| Ammonium Sulfate | 3308 | | | | | | |
| (mg/L) | | | | | | | |
| Potassium Nitrate | 808 | | | | | | |
| (mg/L) | | | | | | | |
| Nitsch and Nitsch | 1 | | | | | | 1 |
| (NN) vitamins (ml/L) | | | | | | | |
| 1000X Stock | | | | | | | |
| Solution | | | | | | | |
| (Phytotech | | | | | | | |
| Catalog # N608) | | | | | | | |
| B5 Vitamins (ml/L) | | 1 | 1 | 1 | 1 | 1 | |
| 1000x stock solution | | | | | | | |
| (Phytotech | | | | | | | |
| Catalog # G249) | | | | | | | |
| Ferrous Sulfate | 5 | 5 | 5 | 5 | 5 | | |
| Chelate solution | | | | | | | |
| (100x) ml/L. | | | | | | | |
| (Phytotech | | | | | | | |
| Catalog # F318) | | | | | | | |
| Sucrose g/L | 20 | 20 | 20 | 20 | 20 | 20 | 20 |
| Agar g/L | 7 | 7 | 7 | 7 | 7 | 7 | 7 |
| Phytagel g/L | | | | | | | |
| Coconut water | 100 | 100 | 100 | 100 | 100 | | |
| (Phytotech | | | | | | | |
| Catalog # C195) | | | | | | | |
| Casein Hydrolysate | | | | | | 2 | 2 |
| g/L | | | | | | | |
| Polyvinylpyrrolidone | | | | | 100 | | |
| PVP-10 (mg/L) | | | | | | | |
| 1-Naphthalene acetic | 1 | 1 | 1 | | 1 | | |
| acid (NAA) (mg/L) | | | | | | | |
| 2,4-D (mg/L) | 0.1 | 0.1 | 0.1 | 1.0 | 0.1 | 1 | 1 |
| Kinetin (mg/L) | 0.5 | 0.5 | 0.5 | 0.5 | 0.5 | 1 | 1 |

| | VM1443 | VM1448 | DC1152 | VM1707 | VM1516 |
|---|---|---|---|---|---|
| MS salts (g/L) | | | 4.33 | 4.33 | 4.33 |
| (Phytotech | | | | | |
| Catalog # M524) (g/L) | | | | | |
| Gamborg's B5 Salts | | | | | |
| (g/L) | | | | | |
| (Phytotech Catalog # | | | | | |
| G768) | | | | | |
| MS basal salts no | 0.788 | 0.788 | | | |
| nitrogen (g/L) | | | | | |
| (Phytotech | | | | | |
| Catalog # M531) | | | | | |
| B5 major salts modified | | | | | |
| (ml/L) | | | | | |
| (20x) (EPS 000210 - | | | | | |
| Table 6) | | | | | |
| B5 minor salts (ml/L) | | | | | |
| (1000x) (EPS 0004 - | | | | | |
| Table 7) | | | | | |
| MS vitamins (mL/L) | | | | | |
| 1000X Stock | | | | | |
| Solution | | | | | |
| (Phytotech | | | | | |
| Catalog # M533) | | | | | |
| Ammonium Sulfate | 1321 | 134 | | | |
| (mg/L) | | | | | |
| Potassium Nitrate (mg/L) | 808 | 808 | | | |
| NN vitamins (ml/L) | 1 | 1 | 1 | 1 | 1 |
| 1000X Stock | | | | | |
| Solution | | | | | |
| (Phytotech | | | | | |
| Catalog # N608) | | | | | |
| B5 Vitamins (ml/L) | | | | | |
| 1000x stock solution | | | | | |
| (Phytotech | | | | | |
| Catalog # G249) | | | | | |
| Ferrous Sulfate Chelate | 5 | 5 | | 5 | 5 |
| solution (100x) ml/L. | | | | | |
| (Phytotech | | | | | |
| Catalog # F318) | | | | | |
| Sucrose g/L | 20 | 20 | 30 | 20 | 20 |
| Agar g/L | 7 | 7 | | 7 | 7 |
| Phytagel g/L | | | 2.5 | | |
| Coconut water | 100 | 100 | | | 100 |
| (Phytotech | | | | | |
| Catalog # C195) | | | | | |
| Casein Hydrolysate g/L | | | | | |
| Polyvinylpyrrolidone | | | | | |
| PVP-10 (mg/L) | | | | | |
| NAA (mg/L) | 1 | 1 | | 1 | 1 |
| 2,4-D (mg/L) | 0.1 | 0.1 | 1.5 | 0.1 | 0.1 |
| Kinetin (mg/L) | 0.5 | 0.5 | | 0.5 | 0.5 |
| | VM1570 | VM1571 | VM1572 | VM1573 | VM1727 |
| MS salts (g/L) | | | | | 4.33 |
| (Phytotech | | | | | |
| Catalog # M524) (g/L) | | | | | |
| Gamborg's B5 Salts | 3.1 | 3.1 | 3.1 | 3.1 | |
| (g/L) | | | | | |
| (Phytotech Catalog # | | | | | |
| G768) | | | | | |
| MS basal salts no | | | | | |
| nitrogen (g/L) | | | | | |
| (Phytotech | | | | | |
| Catalog # M531) | | | | | |
| B5 major salts modified | | | | | |
| (ml/L) | | | | | |
| (20x) (EPS 000210 - | | | | | |
| Table 6) | | | | | |
| B5 minor salts (ml/L) | | | | | |
| (1000x) (EPS 0004 - | | | | | |
| Table 7) | | | | | |
| MS vitamins (mL/L) | | | | | |
| 1000X Stock | | | | | |
| Solution | | | | | |
| (Phytotech | | | | | |
| Catalog # M533) | | | | | |
| Ammonium Sulfate | | | | | |
| (mg/L) | | | | | |
| Potassium Nitrate (mg/L) | | | | | |
| NN vitamins (ml/L) | | | | | 1 |
| 1000X Stock | | | | | |
| Solution | | | | | |
| (Phytotech | | | | | |
| Catalog # N608) | | | | | |
| B5 Vitamins (ml/L) | 1 | 1 | 1 | 1 | |
| 1000x stock solution | | | | | |
| (Phytotech | | | | | |
| Catalog # G249) | | | | | |
| Ferrous Sulfate Chelate | | | | | 5 |
| solution (100x) ml/L. | | | | | |
| (Phytotech | | | | | |
| Catalog # F318) | | | | | |
| Sucrose g/L | 20 | 20 | 20 | 20 | 20 |
| Agar g/L | 7 | 7 | 7 | 7 | 7 |
| Phytagel g/L | | | | | |
| Coconut water | | | | 100 | 100 |
| (Phytotech | | | | | |
| Catalog # C195) | | | | | |
| Casein Hydrolysate g/L | 2.0 | 2.0 | 2.0 | 2.0 | |
| Polyvinylpyrrolidone | | | | | |
| PVP-10 (mg/L) | | | | | |
| NAA (mg/L) | | 0.1 | 0.5 | | 1 |
| 2,4-D (mg/L) | 2.0 | 1.0 | 1.0 | 1.0 | 0.5 |
| Kinetin (mg/L) | 1.0 | 1.0 | 1.0 | 1.0 | 0.5 |

TABLE 2
Composition of media for suspension cultures

| Components | VM1799 | VM1831 | VM1901 | VM1933 | VM3071 | VM3072 | VM3103 |
|---|---|---|---|---|---|---|---|
| MS salts g/L | 4.33 | 4.33 | 4.33 | 4.33 | 4.33 | 4.33 | 4.33 |
| (Phytotech | | | | | | | |
| Catalog # M524) | | | | | | | |
| (g/L) | | | | | | | |
| NN vitamins ml/L | 1 | 1 | 1 | 1 | 1 | 1 | 1 |
| 1000X Stock | | | | | | | |
| Solution | | | | | | | |
| (Phytotech | | | | | | | |
| Catalog # N608) | | | | | | | |
| Coconut water | | 100 | 100 | 100 | 100 | 100 | 100 |
| ml/L | | | | | | | |
| (Phytotech | | | | | | | |
| Catalog # C195) | | | | | | | |
| Sucrose g/L | 30 | 20 | 20 | 30 | 30 | 30 | 30 |
| Ferrous Sulfate | | 5 | | 5 | 5 | 5 | 5 |
| Chelate solution | | | | | | | |
| (100x) ml/L. | | | | | | | |
| (Phytotech | | | | | | | |
| Catalog # F318) | | | | | | | |
| IAA (mg/L) | | | | | | | |
| NAA(mg/L) | | 1.0 | 1.0 | 1.0 | 1.0 | | |
| IBA (mg/L) | | | | | 0.1 | 1.0 | 2.0 |
| 2,4-D (mg/L) | 1.5 | 0.1 | 0.1 | 0.1 | | | |
| Kinetin (mg/L) | 0.5 | 0.5 | 0.5 | 0.5 | 0.5 | 0.5 | 0.5 |

| Components | VM3014 | VM3110 | VM3111 | DC1151 |
|---|---|---|---|---|
| MS salts g/L | 4.33 | 4.33 | 4.33 | 4.3 |
| (Phytotech | | | | |
| Catalog # M524) (g/L) | | | | |
| NN vitamins ml/L | 1 | 1 | 1 | 1 |
| 1000X Stock | | | | |
| Solution | | | | |
| (Phytotech | | | | |
| Catalog # N608) | | | | |
| Coconut water ml/L | 100 | 100 | 100 | |
| (Phytotech | | | | |
| Catalog # C195) | | | | |
| Sucrose g/L | 30 | 20 | 20 | 30 |
| Ferrous Sulfate Chelate solution | 5 | 5 | | |
| (100x) ml/L. (Phytotech | | | | |
| Catalog # F318) | | | | |
| IAA (mg/L) | | 1.0 | 2.0 | |
| NAA(mg/L) | | | | |
| IBA (mg/L) | 3.0 | | | |
| 2,4-D (mg/L) | | | | 1.5 |
| Kinetin (mg/L) | 0.5 | 0.5 | 0.5 | |

The present disclosure may be embodied in other specific forms without departing from its spirit or essential characteristics. The described embodiments are to be considered in all respects only as illustrative and not restrictive. The scope of the disclosure is, therefore, indicated by the appended claims rather than by the foregoing description. All changes which come within the meaning and range of equivalency of the claims are to be embraced within their scope.

### REFERENCES

1. Maatta-Riihinen, K; Kahkonen, M; Toronnen, A R; Hainonen, I M; Catechins and Procyanidins in Berries of Vaccinium Species and Their Antioxidant Activity. J Agric. Food Chem. 2005. 53: 8485-8491.

2. Gu, L; Kelm, M A; Hammerstone, J F; Beecher, G; Holden, J; Haytowitz, D; Prior, R L Screening of foods containing proanthocyanidins and their structural characterization using LC-MS/MS and thiolytic degradation. J. Agric. Food Chem. 2003. 51: 7513-7521.

3. Morimoto, S; Nonaka, G I; Nishioka, I Tannins and related compounds. LX. Isolation and characterization of proanthocyanidins with a double-linked unit from Vaccinium Vitis-idaea L. Chem. Pharm. Bull. 1988. 36: 33-38.

4. Foo, L Y; Lu, Y; Howell, A B; Vorsa, N. A-type proanthocyanidin trimers from cranberry that inhibit adherence of uropathogenic P-fimbriated Escherichia coli. J. Nat. Prod. 2000. 63: 1225-1228.

5. Prior, R L; Lazarus, S A; Cao, G; Muccitelli, H; Hammerstone, J F Identification of procyanidins and anthocyanins in blueberries and cranberries (Vaccinium spp.) using high performance liquid chromatography/mass spectrometry. J. Agric.Food Chem. 2001. 49: 1270-1276.

6. Schmidt, B M.; Howell, A B.; McEniry, B; Knight, C T; Seigler, D; Erdman, J W, Jr.; Lila, M A. Effective separation of potent antiproliferation and antiadhesion components from wild blueberry (Vaccinium angustifolium Ait.) fruits. J. Agric. Food Chem. 2004. 52: 6433-6442.

7. Nowack, R; Schmitt, W. Cranberry juice for prophylaxis of urinary tract infections conclusions from clinical experience and research. Phytomedicine. 2008. 15 (9): 653-67.

8. Foo, L Y; Lu, Y; Howell, A B.; Vorsa, N. The structure of cranberry proanthocyanidins which inhibit adherence of uropathogenic P-fimbriated Escherichia coli in vitro. Phytochemistry 2000. 54: 173-81.

9. Madhavi, D L; Bomser, J; Smith, M A L. Singletary K. Isolation of bioactive constituents from Vaccinium myrtillus fruit and cell culture. Plant Sci 1998. 131: 95-103.

10. Hokkanenm J; Mattila, S; Jaakola, L; Pirttila, A M; Tolonen, A. Identification of Phenolic Compounds from Lingonberry (Vaccinium vitis-idaea L.), Bilberry (Vaccinium myrtillus L.) and Hybrid Bilberry (Vaccinium x intermedium Ruthe L.) Leaves. J. Agric. Food Chem. 2009. 57: 9437-9447.

11. Slinkard, K; Singleton, V L. Total Phenol Analysis:Automation and Comparison with Manual Methods. American Journal of Enology and Viticulture 1977, 28: 49-55.

1. A cell culture, comprising:
a plurality of friable Vaccinium myrtillus cells in a suspension cell culture, the cells being derived from one or more of:
a hypocotyl, a cotyledon, a leaf section, a stem section, or a root section of a seedling; or
a berry, a stem section including a node or an internode, or a leaf section of a mature plant,
wherein the plurality of Vaccinium myrtillus cells are selected to be capable of obtaining a packed cell volume of at least 55% in 7 days of growth, and
wherein at least 5% of a dry mass of the plurality of Vaccinium myrtillus cells is comprised of procyanidins.

2. The cell culture of claim 1, wherein the berry includes a berry skin.

3. The cell culture of claim 1, wherein at least 10%, 15%, or 20% of a dry mass of the plurality of Vaccinium myrtillus cells is comprised of polyphenols.

4. The cell culture of claim 1, wherein at least 7.5% of the dry mass of the plurality of Vaccinium myrtillus cells is comprised of procyanidins.

5. The cell culture of claim 1, wherein at least 10% of the dry mass of the plurality of Vaccinium myrtillus cells is comprised of procyanidins.

6. The cell culture of claim 1, wherein at least 15% of the dry mass of the plurality of Vaccinium myrtillus cells is comprised of procyanidins.

7. The cell culture of claim 1, wherein the dry mass of the plurality of Vaccinium myrtillus cells comprises less than 0.5% anthocyanin.

8. The cell culture of claim 1, wherein the dry mass of the plurality of Vaccinium myrtillus cells comprises less than 0.1% anthocyanin.

9. The cell culture of claim 1, wherein the dry mass of the plurality of Vaccinium myrtillus cells comprises less than 0.01% anthocyanin.

10. The cell culture of claim 1, wherein the dry mass of the plurality of Vaccinium myrtillus cells comprises less than 0.001% anthocyanin.

11. The cell culture of claim 1, wherein the cells in the suspension cell culture are derived from a cell callus of Vaccinium myrtillus.

12. The cell culture of claim 1, wherein the suspension cell culture includes a granular suspension of cells.

13. The cell culture of claim 1, wherein the suspension cell culture includes a fine suspension of cells.

14. The cell culture of claim 1, wherein the plurality of friable Vaccinium myrtillus cells in the suspension cell culture are derived from an edible plant part.

15. The cell culture of claim 14, wherein the edible plant part is one or more of a leaf part or a berry part.

16. A cell culture, comprising:
a plurality of friable Vaccinium myrtillus cells in a suspension cell culture, the cells being derived from an edible plant part,
wherein the plurality of Vaccinium myrtillus cells are selected to be capable of obtaining a packed cell volume of at least 55% in 7 days of growth, and
wherein at least 10%, 15%, or 20% of a dry mass of the plurality of Vaccinium myrtillus cells is comprised of polyphenols and at least 5% of a dry mass of the plurality of Vaccinium myrtillus cells is comprised of procyanidins.

17. The cell culture of claim 16, wherein the edible plant part is one or more of a leaf or a berry.

18. The cell culture of claim 16, wherein the berry includes a berry skin.

19. A cell culture, comprising:
a plurality of friable Vaccinium myrtillus cells in a suspension cell culture, the cells being derived from at least a portion of a berry,
wherein the plurality of Vaccinium myrtillus cells are selected to be capable of obtaining a packed cell volume of at least 55% in 7 days of growth, and
wherein at least 10%, 15%, or 20% of a dry mass of the plurality of Vaccinium myrtillus cells is comprised of polyphenols and at least 5% of a dry mass of the plurality of Vaccinium myrtillus cells is comprised of procyanidins.

20. The cell culture of claim 19, wherein the berry portion includes a berry skin.

21-75. (canceled)
